(19) United States
(12) Patent Application Publication
(10) Pub. No.: US 2018/0128846 A1
(43) Pub. Date: May 10, 2018

(54) INVENTORY ALERT SYSTEM FOR LABORATORIES

(71) Applicant: Roche Diagnostics Operations, Inc., Indianapolis, IN (US)

(72) Inventors: Thomas Baeriswyl, Kriens (CH); David Egan, Zurich (CH)

(73) Assignee: Roche Diagnostics Operations, Inc., Indianapolis, IN (US)

(21) Appl. No.: 15/795,418

(22) Filed: Oct. 27, 2017

(30) Foreign Application Priority Data
Nov. 4, 2016 (EP) 16197229.4

(51) Int. Cl. G01N 35/00; G06F 19/00; G06Q 10/06

(52) U.S. Cl. CPC G01N 35/00732; G01N 35/00871; G06Q 10/087; G06Q 10/06; G06F 19/366

(57) ABSTRACT

Disclosed herein is a system and computer implemented method for detecting extraordinary demand of a consumable(s) for processing biological samples by laboratory instrument(s) in a plurality of laboratories based on exceptional deviations from usual demand levels of trigger laboratories in the proximity of a target laboratory.

### CROSS-REFERENCE TO RELATED APPLICATIONS

This application is a continuation of EP 16197229.4, filed Nov. 4, 2016, which is hereby incorporated by reference.

### BACKGROUND

The present disclosure generally relates to an inventory alert system for laboratories and, in particular, to laboratories comprising at least one laboratory instrument configured to carry out at least one processing step of a biological sample, in particular a laboratory test. The present disclosure further relates to a computer implemented method for alerting laboratories of consumable demand deviation.

In vitro diagnostic testing has a major effect on clinical decisions, providing physicians with pivotal information. Particularly, there is great emphasis on providing quick and accurate test results in critical care settings.

Diagnostic testing makes use of various consumables such as reagents, quality control material such as positive and negative controls, calibrator material, microplates/microwell plates, measurement cuvettes, sample tubes, pipetting tips, etc. In order to be able to carry out laboratory tests, it is therefore of utmost importance that laboratories have all required consumables available.

Existing inventory management systems for diagnostic laboratories have fixed configured values for the maximum amount of each consumable that is ordered and/or stored in a laboratory (stock). The customer enters a value, and the system uses this value until the system is re-configured. Orders of consumables are based on the current stock and a (usually static) maximum amount of the consumable to be stocked, an order being triggered when the current stock drops below a set threshold value.

Widespread occurrence of an infectious disease or even an increased likelihood of the same in a region at a particular time cause laboratories to perform more of a specific laboratory measurement of a biological sample (laboratory test) than they would normally expect based on historical data or normal/predicable seasonal fluctuations. Therefore, there is a risk that laboratories run out of consumables during periods of peak consumption which would lead to delays in obtaining laboratory measurement results.

In order to reduce this risk of running out of consumables, currently laboratories increase their stock to cover even more than the expected need for a particular consumable, in an attempt to buffer unpredictable situations.

However, storage space needs to be used to its utmost efficiency as it is coupled with both set-up and recurring costs. This applies even more in case of storage of consumables such as reagents which need to be kept in a defined temperature range, e.g. in a refrigerator.

Furthermore, a stock that constantly exceeds the demand/usage (by a safety margin) inevitably leads to waste, as most of the consumables required for laboratory tests have a strict expiration date.

Therefore, there is a need for an inventory system which ensures that all consumable(s) are always available for all required laboratory tests while avoiding use of unnecessary storage space or waste consumables by too large of a reserve stock of consumables, even when demand deviates from normal/expected patterns of demand.

### SUMMARY

According to the present disclosure, a method and an inventory alert system for laboratories is presented. The inventory alert system can comprise a plurality of laboratories comprising at least one triggering laboratory and at least one target laboratory. Each laboratory can comprise at least one laboratory instrument configured to carry out at least one processing step of a biological sample and a consumable required to carry out the processing step. The inventory alert system can also comprise a control unit communicatively connected to each of the plurality of laboratories by a communication network. The inventory alert system is configured to: determine a usual demand level T of the consumable required to carry out the processing step by the corresponding triggering laboratory, process actual usage data X of the consumable required to carry out the processing step by the corresponding triggering laboratory, determine a deviation D of the actual usage data X from the usual demand level T of the corresponding triggering laboratory, calculate an alert triggering factor F of each deviation D that exceeds an exceptional deviation threshold EDT, and alert the target laboratory when an aggregation Fₜₒₜ of each alert triggering factor F corresponding to each triggering laboratory in the proximity P of the target laboratory exceeds an alert threshold AT.

Accordingly, it is a feature of the embodiments of the present disclosure to provide for an inventory system which ensures that all consumable(s) are always available for all required laboratory tests while avoiding use of unnecessary storage space or waste consumables by too large of a reserve stock of consumables, even when demand deviates from normal/expected patterns of demand. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a schematic diagram of the inventory alert system comprising a plurality of laboratories communicatively connected with a control unit by a communication network according to an embodiment of the present disclosure.

FIG. 2 illustrates a flowchart depicting a method for alerting laboratories, respectively the processing steps the inventory alert system is configured to carry out according to an embodiment of the present disclosure.

FIG. 3 illustrates a chart showing actual usage data X; usual demand level T of the consumable as well as exceptional deviation threshold EDT of a particular consumable required to carry out a processing step of biological sample by a laboratory according to an embodiment of the present disclosure.

FIG. 4 illustrates an example of geographic distribution of a plurality of laboratories of the disclosed inventory alert system, the plurality of laboratories comprising at least one triggering laboratory and at least one target laboratory according to an embodiment of the present disclosure.

FIG. 5 illustrates a flowchart depicting a further embodiment of the disclosed method for alerting laboratories, respectively the processing steps the inventory alert system is configured to carry out, further comprising the calculation of an expected demand deviation of the consumable for the target laboratory.

FIG. 6 illustrates a flowchart depicting a further embodiment of the disclosed method for alerting laboratories, respectively the processing steps the inventory alert system is configured to carry out, further comprising generation of an order sheet or automatic order of the corresponding consumable based on the expected demand deviation of the consumable for the target laboratory.

FIG. 7 illustrates a schematic diagram of a further embodiment of the inventory alert system, further comprising an input device communicatively connected to the control unit.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

Disclosed herein is a system and computer implemented method for detecting extraordinary demand of a consumable(s)—for processing biological samples by laboratory instrument(s)—based on exceptional deviations from usual demand levels of trigger laboratories in the proximity of a target laboratory.

According to embodiments herein disclosed, the inventory alert system for laboratories can comprise a plurality of laboratories and a control system communicatively connected thereto. The plurality of laboratories can comprise at least one triggering laboratory and at least one target laboratory, while each laboratory comprising at least one laboratory instrument configured to carry out at least one processing step of a biological sample. At least one consumable can be required to carry out the processing step of a biological sample.

The inventory alert system can be configured in a first step to determine a usual demand level T of the consumable required to carry out the processing step of a biological sample by the corresponding triggering laboratory.

In a further step, the inventory alert system can process actual usage data X of the consumable required to carry out the processing step of a biological sample by the respective triggering laboratory. Based on this data, the inventory alert system can determine a deviation D of the actual usage data X from the usual demand level T of the corresponding triggering laboratory.

An alert triggering factor F can then be calculated for each deviation D that exceeds an exceptional deviation threshold. These deviations can be called exceptional deviations. Deviations below the exceptional deviation threshold can be considered normal fluctuations in the actual usage of the consumables and may not be considered for triggering any alerts.

Target laboratory(s) can be alerted when the aggregation of each alert triggering factor F corresponding to each triggering laboratory in the proximity of the target laboratory exceeds an alert threshold AT.

According to embodiments herein disclosed, the inventory alert system respectively the disclosed alert method can be advantageous on multiple levels of configurability and adaptability are enabled thereby: by an exceptional deviation threshold, normal consumable demand fluctuations can be filtered out in a manner specific to each triggering laboratory; by a proximity function—and its weighting(s), the relevance (or influence) of each triggering laboratory can be fine-tuned to the specific target laboratory; and by setting an appropriate alert threshold of aggregated triggering factors, false triggers due to alert trigger factors of low relevance can be filtered out.

In some embodiments, the usual demand level of the consumable required to carry out the processing step of a biological sample can be determined based on an average and/or median number of conducted and/or ordered processing step(s) per defined period corresponding to the respective triggering laboratory and/or determined based on historical data comprising one or more of: a seasonal variation; a schedule-based variation; an event-linked variation and a trend associated variation; geographically influenced variation.

In some embodiments, the alert triggering factor can be a function of one or more of: the usual demand level, the exceptional deviation and the proximity P of the triggering laboratory and the target laboratory.

In some embodiments, the proximity P can be a function. In some embodiments, the proximity P can be a weighted function.

In some embodiments, alerting the target laboratory can comprise calculation of an expected demand deviation of the consumable for the target laboratory. The expected demand deviation can be a function of the usual demand level T of the target laboratory and/or a function of each alert triggering factor F corresponding to each triggering laboratory in the proximity P of the target laboratory and/or a function of the deviation D of each triggering lab.

In some embodiments, alerting the target laboratory can comprise generation of an order sheet of the corresponding consumable based on the expected demand deviation of the consumable for the target laboratory and the actual inventory of the consumable in the target laboratory and/or automatically ordering the corresponding consumable based on the order sheet for target laboratory.

In some embodiments, the at least one laboratory instrument can comprise at least one analytical instrument. The at least one processing step of a biological sample can comprise at least one diagnostic test of the biological sample for obtaining a measurement value from biological sample.

In some embodiments, the at least one laboratory instrument can comprise at least one pre-analytical instrument. The at least one processing step of a biological sample can comprise the centrifugation, aliquotation, analyte isolation of an analyte from the biological sample.

As used herein, the terms ‘comprises,’ ‘comprising,’ ‘includes,’ ‘including,’ ‘has,’ ‘having’ or any other variation thereof, can be intended to cover a non-exclusive inclusion of features.

An ‘analysis system’ as used herein can comprise a control unit operatively coupled to one or more analytical; pre- and post-analytical devices. The control unit can control the devices. In addition, the control unit may be operable to evaluate and/or process gathered analysis data, to control the loading, storing and/or unloading of samples and/or consumables to and/or from any one of the devices, to initialize an analysis or hardware or software operations of the analysis system used for preparing the samples, sample tubes or reagents for the analysis and the like.

The term ‘analyte’ can be a component of a sample to be analyzed, e.g. molecules of various sizes, ions, proteins, metabolites and the like. Information gathered on an analyte may be used to evaluate the impact of the administration of drugs on the organism or on particular tissues or to make a diagnosis. Thus ‘analyte’ is a general term for substances for which information about presence and/or concentration is intended. Examples of analytes can be, for example, glucose, coagulation parameters, endogenic proteins (e.g. proteins released from the heart muscle), metabolites, nucleic acids and so on.

The term ‘analytical data’ as used herein can encompass any data that can be descriptive of a result of a measurement of a biological sample. In the case of a calibration, the analytical data can comprise the calibration result, i.e. calibration data. In one embodiment, the analytical data can comprise an identifier of the sample for which the analysis has been performed and data can be descriptive of a result of the analysis, such as measurement data.

The term ‘laboratory instrument’ as used herein can encompass any apparatus or apparatus component operable to execute one or more processing steps/workflow steps on one or more biological samples and/or one or more reagents. The expression ‘processing steps’ can thereby refer to physically executed processing steps such as centrifugation, aliquotation, analyte isolation, sample analysis and the like. The term ‘instrument’ can cover pre-analytical instruments, post-analytical instruments and also analytical instruments.

The term ‘analyzer’/‘analytical instrument’ as used herein can encompass any apparatus or apparatus component configured to obtain a measurement value. An analyzer can determine via various chemical, biological, physical, optical or other technical procedures a parameter value of the sample or a component thereof. An analyzer may measure the parameter of the sample or of at least one analyte and return the obtained measurement value. The list of possible analysis results returned by the analyzer can comprise, without limitation, concentrations of the analyte in the sample, a digital (yes or no) result indicating the existence of the analyte in the sample (corresponding to a concentration above the detection level), optical parameters, DNA or RNA sequences, data obtained from mass spectroscopy of proteins or metabolites and physical or chemical parameters of various types. An analytical instrument may comprise units assisting with the pipetting, dosing, and mixing of samples and/or reagents. The analyzer may comprise a reagent holding unit for holding reagents to perform the assays. Reagents may be arranged, for example, in the form of containers or cassettes containing individual reagents or group of reagents, placed in appropriate receptacles or positions within a storage compartment or conveyor. It may comprise a consumable feeding unit or consumable loading and unloading unit. The analyzer may comprise a process and detection system whose workflow can be optimized for certain types of analysis. Examples of such analyzer can be clinical chemistry analyzers, coagulation chemistry analyzers, immunochemistry analyzers, urine analyzers, nucleic acid analyzers, used to detect the result of chemical or biological reactions or to monitor the progress of chemical or biological reactions.

The term ‘point of care analyzer’ as used herein can encompass any analyzer used in a point of care environment, such as (but not limited to) blood glucose testing, coagulation testing, blood gas and electrolytes analysis, urinalysis, cardiac markers analysis, hemoglobin diagnostics, infectious disease testing and cholesterol screening. Results may be viewed directly on the point of care (POC) analyzer(s) or may be sent to the POC system and displayed in a Laboratory Middleware or a Laboratory Information System (LIS) with central lab results, or alongside imaging results in a Hospital Information System (HIS).

The term ‘analysis’ or ‘analytical test’ as used herein can encompass a laboratory procedure characterizing a parameter of a biological sample for qualitatively assessing or quantitatively measuring the presence or amount or the functional activity of an analyte.

The term ‘pre-analytical instrument’ as used herein can encompass any apparatus or apparatus component that can be configured to perform one or more pre-analytical processing steps/workflow steps comprising—but not limited to—centrifugation, analyte isolation, resuspension (e.g. by mixing or vortexing), capping, decapping, recapping, sorting, tube type identification, sample quality determination and/or aliquotation steps. The processing steps may also comprise adding chemicals or buffers to a sample, concentrating a sample, incubating a sample, and the like.

The term ‘post-analytical instrument’ as used herein can encompass any apparatus or apparatus component that can be configured to perform one or more post-analytical processing steps/workflow steps comprising—but not limited to—sample unloading, transport, recapping, decapping, temporary storage/buffering, archiving (refrigerated or not), retrieval and/or disposal.

The term ‘communication network’ as used herein can encompass any type of wireless network, such as a WIFI, GSM, UMTS or other wireless digital network or a cable based network, such as Ethernet or the like. In one embodiment, the communication network can implement the Internet protocol (IP). For example, the communication network comprises a combination of cable-based and wireless networks.

The term ‘control unit’ as used herein can encompass any physical or virtual data processing device. In some embodiments, the control unit may be integral with a data management unit, may be comprised by a server computer and/or be part of one laboratory instrument or even distributed across multiple instruments of the laboratory system. The control unit may, for instance, be embodied as a programmable logic controller running a computer-readable program provided with instructions to perform operations. A “data management unit” can be a computing unit for storing and managing data. This may involve data relating to consumable(s) required to carry out processing step(s) on biological samples. The data management unit may be connected to an LIS (laboratory information system) and/or an HIS (hospital information system). The data management unit (DMU) can be a unit within or co-located with an automated system. It may be part of the control unit. Alternatively, the DMU may be a unit remotely located. For instance, it may be embodied in a computer connected via a communication network.

The terms ‘sample’, ‘patient sample’ and ‘biological sample’ can refer to material(s) that may potentially contain an analyte of interest. The patient sample can be derived from any biological source, such as a physiological fluid, including blood, saliva, ocular lens fluid, cerebrospinal fluid, sweat, urine, stool, semen, milk, ascites fluid, mucous, synovial fluid, peritoneal fluid, amniotic fluid, tissue, cultured cells, or the like. The patient sample can be pretreated prior to use, such as preparing plasma from blood, diluting viscous fluids, lysis or the like. Methods of treatment can involve filtration, distillation, concentration, inactivation of interfering components, and the addition of reagents. A patient sample may be used directly as obtained from the source or used following a pretreatment to modify the character of the sample. In some embodiments, an initially solid or semi-solid biological material can be rendered liquid by dissolving or suspending it with a suitable liquid medium. In some embodiments, the sample can be suspected to contain a certain antigen or nucleic acid.

The term ‘user interface’ as used herein can encompass any suitable piece of software and/or hardware for interactions between an operator and a machine, including but not limited to a graphical user interface for receiving as input a command from an operator and also to provide feedback and convey information thereto. Also, a system/device may expose several user interfaces to serve different kinds of users/operators.

The inventory alert system 1 for laboratories respectively the computer implemented method for alerting laboratories may now be described with reference to particular embodiments thereof.

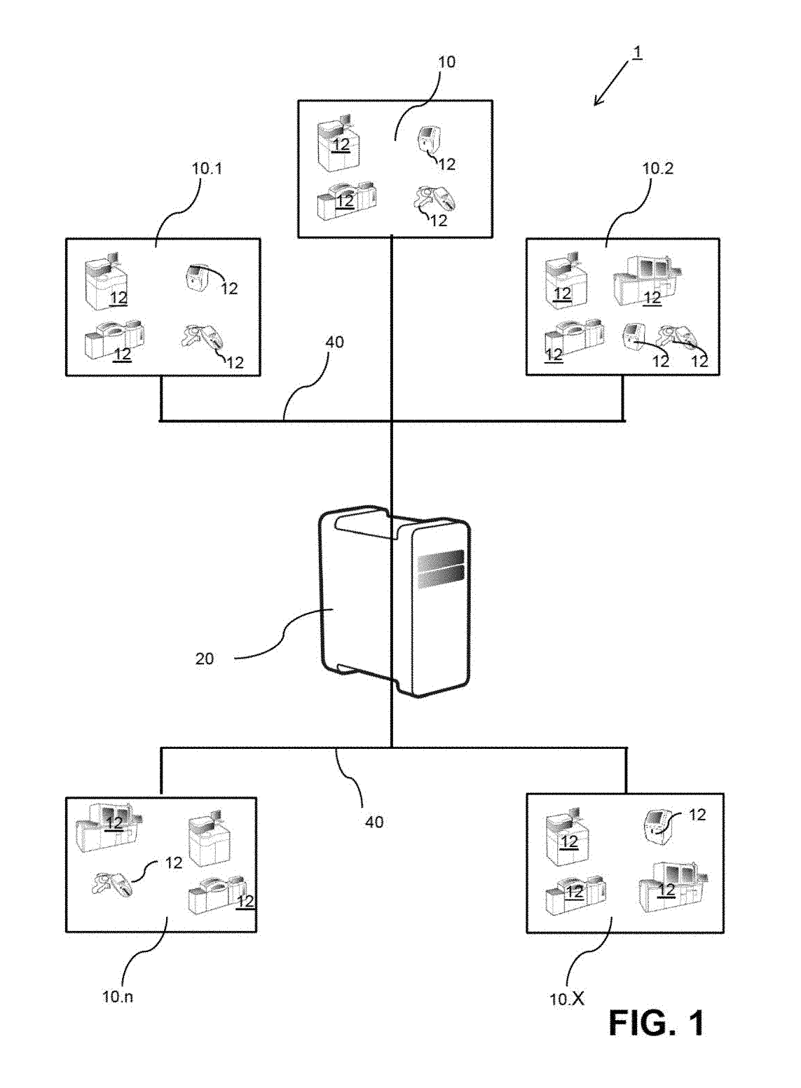

As illustrated on FIG. 1, the inventory alert system 1 for laboratories can comprise a plurality of laboratories 10 communicatively connected with a control unit 20 by a communication network 40. The laboratories 10 may be located in different geographical locations and/or in different areas of one particular location, such as various labs of the same hospital, diagnostic center, etc.

Each laboratory 10 of the plurality of laboratories 10 can comprise at least one laboratory instrument 12 configured to carry out at least one processing step of a biological sample and a consumable required to carry out the processing step. The at least one laboratory instrument 12 may be one or more of a pre-analytical instrument; analytical instrument and/or post-analytical instrument. Correspondingly the at least one processing step of a biological sample can comprise one or more of:

pre-analytical processing step(s), comprising—but not limited to—centrifugation, separation/isolation, resuspension (e.g. by mixing or vortexing), capping, decapping, recapping, sorting, tube type identification, sample quality determination and/or aliquotation steps;

diagnostic test(s) of the biological sample for obtaining a measurement value from biological sample; and/or

post-analytical processing steps, comprising—but not limited to—sample unloading, transport, recapping, decapping, temporary storage/buffering, archiving (refrigerated or not), retrieval and/or disposal.

Corresponding to the processing step of a biological sample, the at least one consumable required to carry out the processing step can comprise—but not limited to—one or more of: reagents; diluents; liquid handling disposables such as pipetting tips; reaction vessels, such as cuvettes, sample plates, microwell plates, and the like.; sample tubes; and/or sample tube racks.

The plurality of laboratories 10 can comprise at least one laboratory which can be a triggering laboratory 10.1-10.n and at least one laboratory which can be a target laboratory 10.X. It can be noted that the same laboratory 10 may be both a triggering laboratory 10.1-10.n and target laboratory 10.X at different points in time.

The at least one triggering laboratory 10.1-10.n and at least one target laboratory 10.X can be communicatively connected to each of the plurality of laboratories 10 by a communication network 40, which may be a wired or wireless communication network or a combination thereof.

FIG. 1 shows one embodiment of the disclosed system 1 wherein the control unit 20 can be a stand-alone, dedicated server computer. Nevertheless, according to various embodiments (not shown on the figures) the control unit 20 may be a virtual processing device comprised by a multi-purpose server computer and/or be part of one laboratory instrument or even distributed across multiple instruments of the laboratory system.

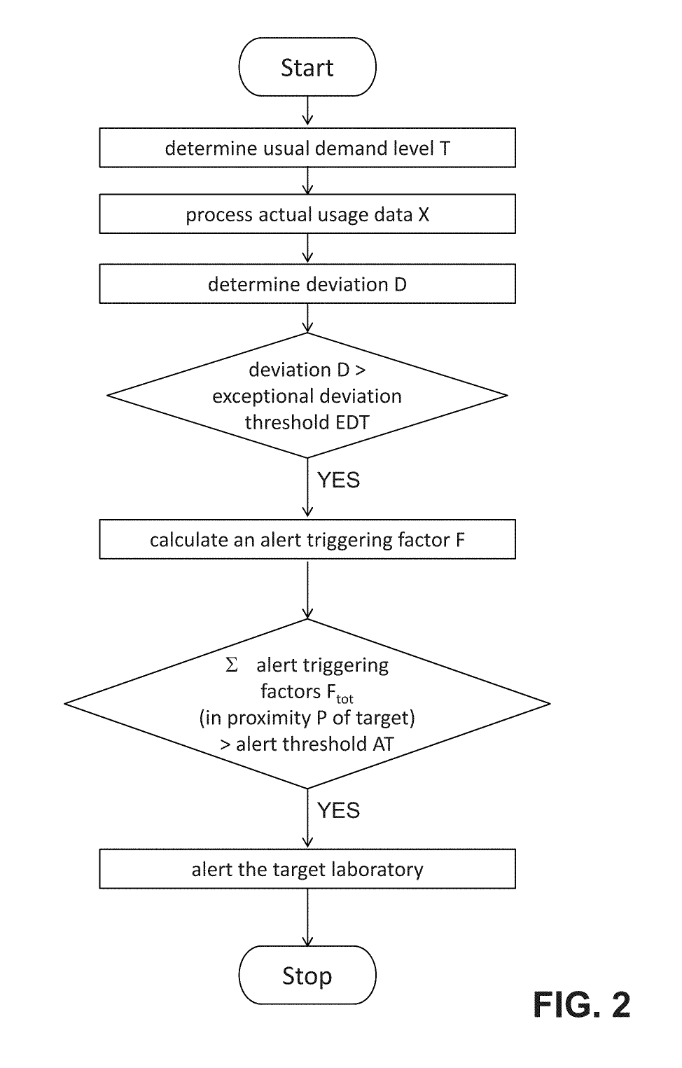

Turning now to FIG. 2, the steps of the disclosed method for alerting laboratories can be described—the method being implemented by a computer system, in particular by the inventory alert system 1 configured to carry out such method steps.

In a first step, the inventory alert system 1 can be configured to determine a usual demand level T of the consumable required to carry out the processing step by the corresponding triggering laboratory 10.1-10.n.

According to an embodiment, the usual demand level T can be determined by the respective triggering laboratory 10.1-10.n itself. In such embodiments, the triggering laboratory 10.1-10.n can be configured to transmit the usual demand level T to the control unit 20 via the communication network 40.

Alternatively or additionally, according to another embodiment, the usual demand level T can be determined by the control unit 20 based on actual usage data received from the corresponding triggering laboratory 10.1-10.n .

The usual demand level T can represent the expected consumption rate of corresponding consumables of a laboratory 10 and can be determined based on an average and/or median number of conducted and/or ordered processing step(s) per defined period corresponding to the respective triggering laboratory 10.1-10.n and/or determined based on historical data comprising one or more of: a seasonal variation; a schedule-based variation; an event-linked variation and a trend associated variation; geographically influenced variation.

A seasonal fluctuation can be a fluctuation of the consumption of a consumable required to carry out a processing step on a biological sample which can be influenced by calendar seasons, e.g. flu season, when certain diagnostic tests are carried out more frequently than for example in summer.

A schedule-based fluctuation can be a fluctuation of the consumption of a consumable required to carry out a processing step on a biological sample which can be influenced by a schedule of, e.g. weekday vs. weekend, day shift/night shift.

An event-linked fluctuation can be a fluctuation of the consumption of a consumable required to carry out a processing step on a biological sample which can be influenced by an particular event, e.g. a major event, such as Olympic games with a known/expected contamination risk leading to increased demand for a test (e.g. increased Zika Virus testing around the world following the 2016 Olympic Games in Rio de Janeiro). A trend associated fluctuation can be a fluctuation of the consumption of a consumable required to carry out a processing step on a biological sample which can be influenced by a long term tendency to perform more and more test of a type, e.g., due to aging population; due to increased % of population with diabetes, and the like.

Geographically influenced fluctuation can be a fluctuation of the consumption of a consumable required to carry out a processing step on a biological sample which can be influenced by the geographical location of the respective triggering laboratory 10.1-10.n, for example, increased Ebola infections in Sub-Saharan Afrika, increased HIV test in 3^{rd} world countries, and the like.

In a following step, the inventory alert system 1 can be configured to process actual usage data X of the consumable required to carry out the processing step by the corresponding triggering laboratory 10.1-10.n in order to determine a deviation D of the actual usage data X from the usual demand level T of the corresponding triggering laboratory 10.1-10.n.

According to one embodiment, the processing of the actual usage data X and/or determination of the deviation D of the actual usage data X from the usual demand level T can be performed by the respective triggering laboratory 10.1-10.n itself. In such embodiments, the triggering laboratory 10.1-10.n can be configured to transmit the deviation D to the control unit 20 via the communication network 40.

Alternatively or additionally, according to another embodiment, the processing of the actual usage data X and/or determination of the deviation D of the actual usage data X from the usual demand level T can be performed by the control unit 20 based on actual usage data X received from the respective triggering laboratory 10.1-10.n via the communication network 40.

According to further embodiments, the usual demand level T and/or actual usage data X can be anonymized to protect the privacy of the plurality of triggering laboratories 10.1-10.n.

After the deviation D is determined, it can be compared to an exceptional deviation threshold EDT. If the deviation D is below the exceptional deviation threshold EDT, the deviation D can be considered as a normal—fluctuation of consumable use and therefore can be filtered out, namely such deviations can be excluded from further consideration. According to embodiments disclosed, the exceptional deviation threshold EDT can be set based on historical data, e.g. as a percentage of the usual demand level T of the consumable; an absolute value; etc. The exceptional deviation threshold EDT can be set such as to filter out the normal fluctuation of consumable use to avoid “false alarms” but at the same ensuring that exceptional deviations can be reliably detected.

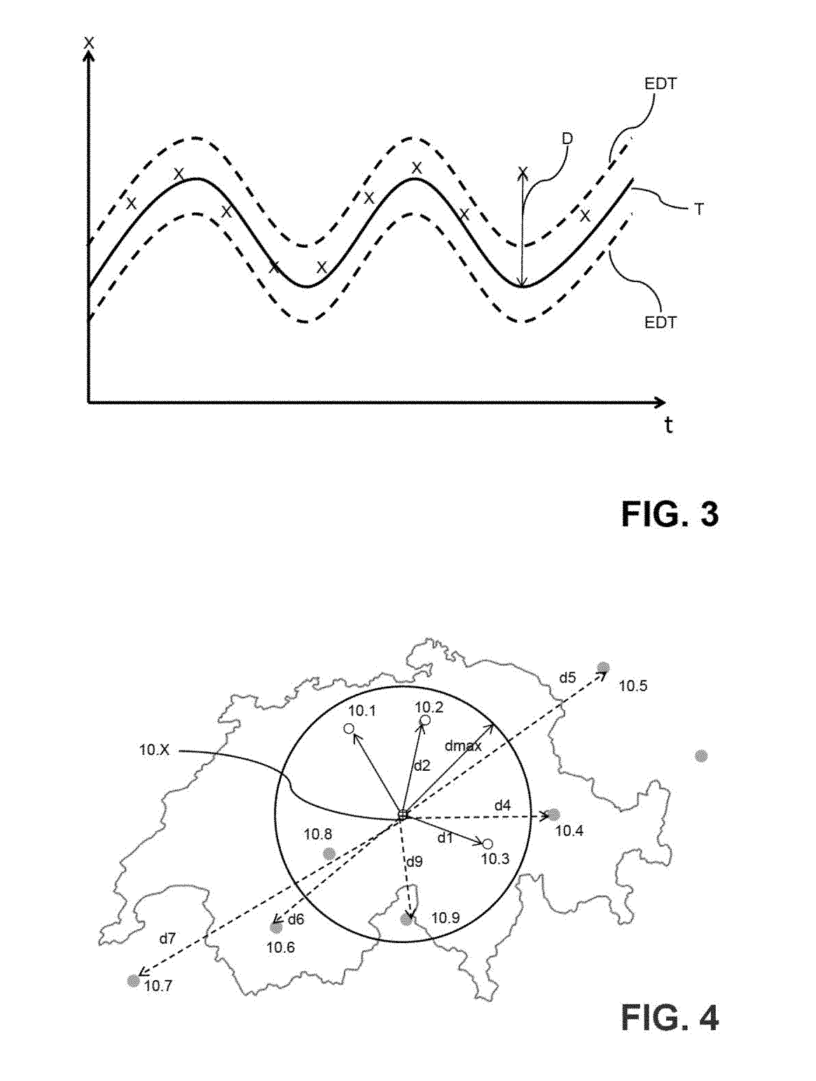

FIG. 3 illustrates an example of how the actual usage data X can be processed in view of the usual demand level T in order to determine the deviation D of the actual usage data X from the usual demand level T, respectively to detect deviations D which can exceed the exceptional deviation threshold EDT. The illustrated fluctuation of the usual demand level T can be for example due to different levels of activities in the laboratory 10 in different times of day (day shift vs. night shift); weekdays vs. weekend; winter (flu season) vs. summer, etc. The aim of comparing the actual usage data X with the usual demand level T can be therefore to detect deviations which exceed/contravene the expected pattern/trend as such can be an indication of a situation (e.g. epidemic) which can lead to increased demand in the target laboratory(s) 10.X in proximity.

Turning back to FIG. 2, if the deviation D is above the exceptional deviation threshold EDT, an alert triggering factor F can be calculated. This alert triggering factor F can be calculated for each and every deviation D that exceeds an exceptional deviation threshold EDT. Herein, each and every deviation D can comprise multiple deviations D originating from the same triggering laboratory 10.1-10.n as well as deviations D originating from the various triggering laboratories 10.1-10.n. The alert triggering factor F can be a function of one or more of: the usual demand level T, the deviation D, and the proximity P of the triggering laboratory 10.1-10.n and the target laboratory 10.X.

According to some embodiments, the alert triggering factor F can be a weighted function. The higher the usual demand level T, the higher its alert-triggering weighting can be and/or the higher the deviation D from the exceptional deviation threshold EDT, the higher its alert-triggering weighting can be.

According to one embodiment, the alert triggering factor F can be calculated by the control unit 20 based on usual demand level T of the triggering laboratories, actual usage data X of the triggering laboratories received via the communication network 40 and the proximity P of the triggering laboratory 10.1-10.n and the target laboratory 10.X.

Alternatively or additionally, the alert triggering factor F can be calculated by the respective triggering laboratory 10.1-10.n itself. In such embodiments, the triggering laboratory 10.1-10.n can be configured to transmit the alert triggering factor F to the control unit 20 via the communication network 40.

Following the calculation of the alert triggering factor F of each deviation D that exceeds an exceptional deviation threshold EDT, each alert triggering factor F corresponding to the triggering laboratories 10.1-10.n in the proximity P of the target laboratory 10.X can be aggregated.

By aggregating only the alert triggering factor(s) F of the triggering laboratories 10.1-10.n in the proximity P of the target laboratory 10.X, the disclosed system/method can aim at filtering out alert triggers which due to their non-proximity may not be relevant to the particular target laboratory 10.X. In other words, the aggregation of select alert triggering factor(s) F can be tailored to the specific target laboratory 10.X.

According to embodiments of the disclosed system/method, the proximity P can be a function, in one embodiment, a weighted function of one or a combination of more of the following parameters:

a geographical distance d1-dn between the triggering laboratory 10.1-10.n and the target laboratory 10.X;

a travelling distance dt (considering a certain transport infrastructure, e.g roads or railway) between the triggering laboratory 10.1-10.n and the target laboratory 10.X;

a travelling time tt between the triggering laboratory 10.1-10.n and the target laboratory 10.X;

a travelling frequency tf between the triggering laboratory 10.1-10.n and the target laboratory 10.X;

a selected geographical and/or political area A.

FIG. 4 illustrates an example of geographical distribution of the plurality of laboratories 10 comprising the triggering laboratories 10.1-10.8 and one target laboratory 10.X. In the embodiment illustrated, the proximity P can be a function, wherein the geographical distance d1-dn between the triggering laboratory 10.1-10.n and the target laboratory 10.X is at least one of the parameters mentioned above. For example, the further the triggering laboratory 10.1-10.n, the lower its contribution to the alert triggering factor F can be.

However, the geographical distance d1-dn between the triggering laboratory 10.1-10.n may not be the only parameter, as exemplified by the laboratories 10.8 respectively 10.9, which while closer to the target laboratory 10.X than the maximum geographical distance dmax, may not be considered for the aggregated alert triggering factor Fₜₒₜ. For example, because laboratory 10.8 did not show a deviation D which exceeded the exceptional deviation threshold EDT, while laboratory 10.9 can be in a different political area (different country) which may have different regulations on certain laboratory tests.

According to some embodiments, the weighted proximity function P can have one or more of the following weights:

a known infectious disease spread pattern overlapping the triggering laboratory 10.1-10.n and the target laboratory 10.X; and/or

a known major event with a known and/or expected elevated infectious disease risk level with known and/or suspected common participants from the geographical area of the triggering laboratory 10.1-10.n and the target laboratory 10.X.

As a next step, the aggregation Fₜₒₜ of each alert triggering factor F corresponding to each triggering laboratory 10.1-10.n in the proximity P of the target laboratory 10.X can be compared to an alert threshold AT.

Fₜₒₜ=Σᵢ₌₁ⁿFi>AT?

If the aggregation Fₜₒₜ exceeds to the alert threshold AT, the target laboratory 10.X can be alerted, and in one embodiment, can be alerted via the communication network 40.

Alerting the target laboratory 10.X can comprise—but is not limited to—transmitting an alert signal to the target laboratory 10.X which can cause a user interface thereof to emit an audible and/or visual indication of the alert, such as a beeping sound drawing attention to an alert message displayed on the user interface of the target laboratory 10.X. Alternatively or additionally, transmitting an alert signal to the target laboratory 10.X can comprise sending an electronic notification such as an email to an email account associated with the target laboratory 10.X, respectively with a user responsible for the consumable inventory associated with the target laboratory 10.X. Alternatively or additionally, transmitting an alert signal to the target laboratory 10.X can comprise interfacing with an inventory management system of thereof, the alert causing the entry corresponding to the particular consumable to be flagged.

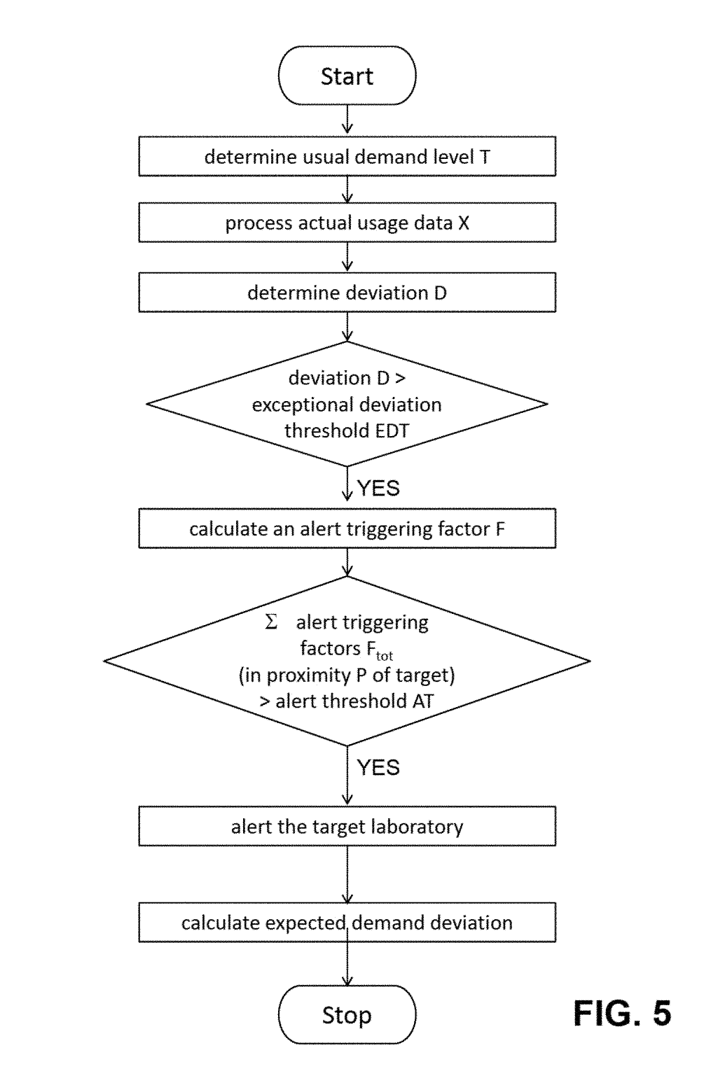

FIG. 5 shows a flowchart of embodiments further comprising the calculation of an expected demand deviation of the consumable for the target laboratory 10.X.

According to embodiments, the expected demand deviation can be calculated as a function of the usual demand level T of the target laboratory 10.X and/or a function of each alert triggering factor F corresponding to each triggering laboratory 10.1-10.n in the proximity P of the target laboratory 10.X and/or a function of the deviation D of each triggering lab. For example, a large deviation D of a consumable at a triggering laboratory 10.1-10.n in the close proximity P (e.g., geographical closeness) can have a larger effect than a comparatively small deviation D of the same consumable at a triggering laboratory 10.1-10.n far from the target laboratory 10.X. As a further example, the same aggregated alert triggering factor Fₜₒₜ can have a greater effect on the expected demand deviation of a large target laboratory 10.X with a high usual demand level T of the corresponding consumable than a small target laboratory 10.X with a low usual demand level T of the same. Also, demand deviation at a specialist laboratory can have a reduced effect on a routine laboratory.

According to some embodiments, alerts and/or the alert triggering factor F can be specific and/or configurable for particular analytic test(s) or type of test(s), such as for example an analytic test for infectious diseases which can be greatly exposed to fluctuations (exceptional deviations) and/or analytic tests which can be time critical (for example, in emergency testing environments).

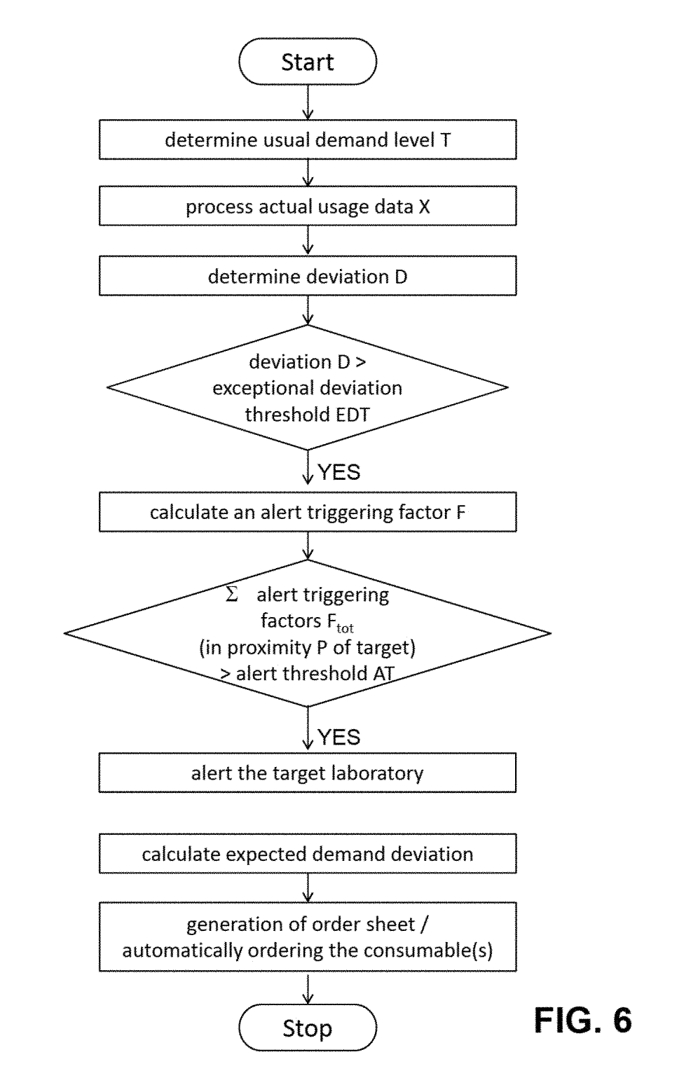

FIG. 6 shows a flowchart of embodiments further comprising generation of an order sheet of the corresponding consumable based on the expected demand deviation of the consumable for the target laboratory 10.X. Furthermore, the order sheet can also be generated based on the actual inventory of the consumable in the target laboratory 10.X. The order sheet may be a conventional list of consumables to be ordered. Alternatively or additionally, the order sheet may be an electronic record to be processed by an inventory management system.

Hence, according to some embodiments, the corresponding consumable can be automatically ordered based on the order sheet for target laboratory 10.X.

The order sheet may be a conventional list of consumables to be ordered. Alternatively or additionally, the order sheet may be an electronic record to be processed by an inventory management system.

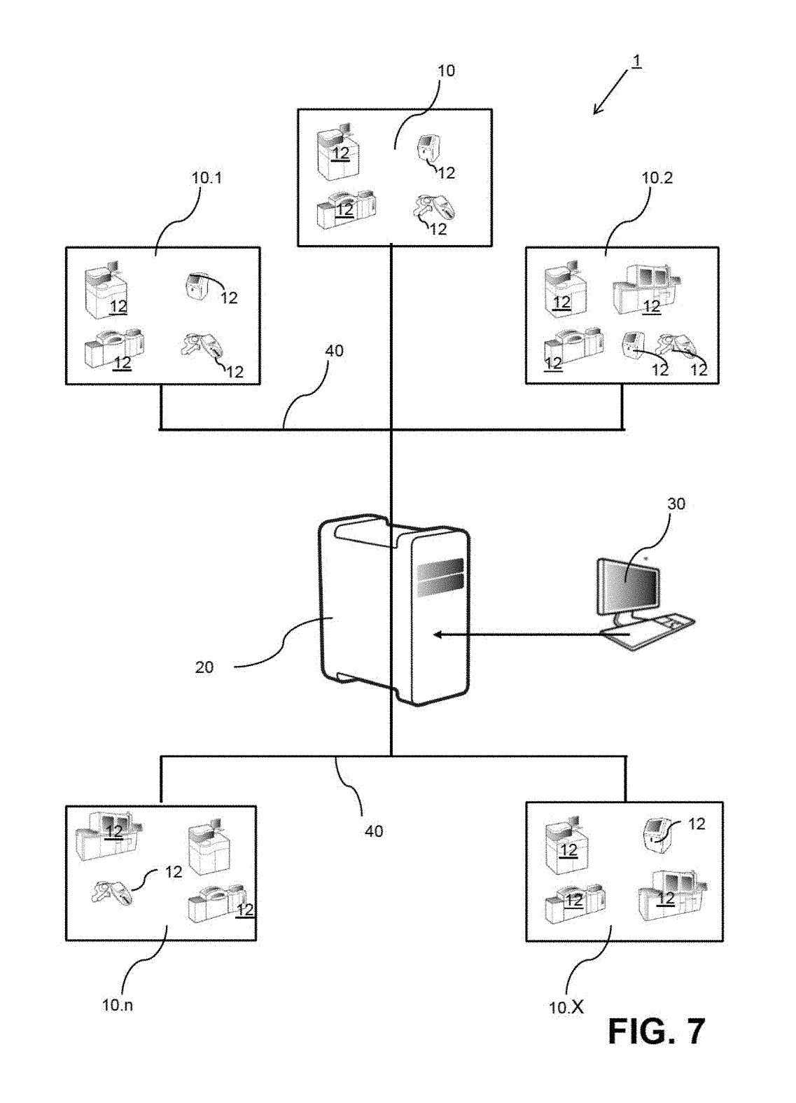

FIG. 7 shows a further embodiment of the inventory alert system 1, further comprising an input device 30 communicatively connected to the control unit 20, either directly or via the communication network 40. The input device 30 may comprise—but is not limited to:

a personal computer, such as a desktop or laptop computer, having a user interface, i.e., a piece of software and/or hardware for interactions between an operator and a machine, including but not limited to a graphical user interface for receiving an input from an operator and also to provide feedback and convey information thereto;

a portable computing device, i.e., a mobile/portable electronic appliance having an interface for communicating with a server computer via a communication network such as, for example, a handheld battery powered mobile appliance, such as a mobile phone, a smart phone, a personal digital assistant (PDA) or another electronic appliance having an interface (wired and/or wireless) for establishing a communication link with a server computer such as over a wireless digital cellular telecommunications network or another communication channel;

a web interface accessible using a web browser for receiving an input from an operator.

Furthermore, the control unit 20 of the embodiments depicted on FIG. 7 can be configured to receive an input from the input device 30. The input received from the input device can be indicative of an accuracy of the calculation of an expected demand deviation. In an embodiment, this input which can be indicative of the accuracy of the calculation can be based on a comparison of previously calculated with the actually experienced deviations of consumable demand/usage by target laboratories 10.X. In a further embodiment, this input which can be indicative of the accuracy of the calculation can be based on an expert review of expected demand deviations.

Based on this input indicative of an accuracy of the calculation of an expected demand deviation, the control unit 20 can be configured to adjust the exceptional deviation threshold EDT and/or the alert threshold AT and/or parameters of the function for calculation of the alert triggering factor F and/or parameters of the function for calculation of the expected demand deviation. By the adjustment, the disclosed system/method can be capable of improving sensitivity and specificity of its alerts as well as the accuracy of the expected demand deviation the longer the system/method is deployed, therefore providing even better and better optimization of the inventories of consumables of the corresponding laboratories 10.1-10.n.

For example, if a comparison shows that the expected demand deviation has been predicted to be far off from the actual deviation reflected by following actual usage data X, an expert review (either by machine learning algorithms or manually) can be triggered to determine which factor, threshold and/or parameter needs to be adjusted to ensure better predictions in the future.

In summary, the disclosed system/method can be advantageous as it can allow multiple levels of configurability and adaptability:

by the exceptional deviation threshold EDT, normal consumable demand fluctuations can be filtered out in a manner specific to each triggering laboratory 10.1-10.n;

by the proximity function P—and its weighting(s), the relevance (or influence) of each triggering laboratory 10.1-10.n can be fine tuned to the specific target laboratory 10.X; and

by setting an appropriate alert threshold AT of aggregated triggering factors Fₜₒₜ, false triggers due to alert trigger factors of low relevance can be filtered out.

In other words, the disclosed system/method can be adaptable to the specifics of each triggering laboratory 10.1-10.n; of each target laboratory 10.X and of the entire system of a plurality of laboratories 10.

Further disclosed and proposed can be a computer program including computer-executable instructions for performing the disclosed method in one or more of the embodiments enclosed herein when the program can be executed on a computer or computer network. Specifically, the computer program may be stored on a computer-readable data carrier. Thus, specifically, one, more than one or even all of method steps may be performed by using a computer or a computer network, preferably by using a computer program.

Further disclosed and proposed is a computer program product having program code, in order to perform the disclosed method in one or more of the embodiments enclosed herein when the program is executed on a computer or computer network. Specifically, the program code may be stored on a computer-readable data carrier. The product may generally exist in an arbitrary format, such as in a paper format, or on a computer-readable data carrier. Specifically, the computer program product may be distributed over a data network.

Further disclosed and proposed is a data carrier having a data structure stored thereon, which, after loading into a computer or computer network, such as into a working memory or main memory of the computer or computer network, may execute the disclosed method according to one or more of the embodiments disclosed herein. As used herein, a computer program product can refer to the program as a tradable product.

Further disclosed and proposed is a modulated data signal which can contain instructions readable by a computer system or computer network, for performing the method according to one or more of the embodiments disclosed herein.

Referring to the computer-implemented aspects, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. An inventory alert system for laboratories, the inventory alert system comprising:
a plurality of laboratories comprising at least one triggering laboratory and at least one target laboratory, each laboratory comprising at least one laboratory instrument configured to carry out at least one processing step of a biological sample and a consumable required to carry out the processing step; and
a control unit communicatively connected to each of the plurality of laboratories by a communication network, wherein the inventory alert system is configured to:
determine a usual demand level T of the consumable required to carry out the processing step by the corresponding triggering laboratory,
process actual usage data X of the consumable required to carry out the processing step by the corresponding triggering laboratory,
determine a deviation D of the actual usage data X from the usual demand level T of the corresponding triggering laboratory,
calculate an alert triggering factor F of each deviation D that exceeds an exceptional deviation threshold EDT, and
alert the target laboratory when an aggregation Fₜₒₜ of each alert triggering factor F corresponding to each triggering laboratory in the proximity P of the target laboratory exceeds an alert threshold AT.

2. The inventory alert system according claim 1, wherein the usual demand level T of the consumable required to carry out the processing step of a biological sample is determined based on an average and/or median number of conducted and/or ordered processing step(s) per defined period corresponding to the respective triggering laboratory and/or determined based on historical data comprising one or more of: a seasonal variation; a schedule-based variation; an event-linked variation and a trend associated variation; geographically influenced variation.

3. The inventory alert system according to claim 1, wherein the alert triggering factor F is a function of one or more of: the usual demand level T, the deviation D, the proximity P of the triggering laboratory and the target laboratory, and/or the proximity P is a weighted function of one or a combination of more of the following parameters:
a geographical distance d1-dn between the triggering laboratory and the target laboratory,
a travelling distance dt between the triggering laboratory and the target laboratory,
a travelling time tt between the triggering laboratory and the target laboratory,
a travelling frequency tf between the triggering laboratory and the target laboratory, and/or
a selected geographical and/or political area A;
the proximity function P having one or more of the following weights:
a known infectious disease spread pattern overlapping the triggering laboratory and the target laboratory, and/or
a known major event with a known and/or expected elevated infectious disease risk level with known and/or suspected common participants from the geographical area of the triggering laboratory and the target laboratory.

4. The inventory alert system according to claim 1, wherein alerting the target laboratory comprises calculation of an expected demand deviation of the consumable for the target laboratory, the expected demand deviation being a function of the usual demand level T of the target laboratory and/or a function of each alert triggering factor F corresponding to each triggering laboratory in the proximity P of the target laboratory and/or a function of the deviation D of each triggering lab.

5. The inventory alert system according to claim 4, wherein alerting the target laboratory comprises generation of an order sheet of the corresponding consumable based on the expected demand deviation of the consumable for the target laboratory and the actual inventory of the consumable in the target laboratory and/or automatically ordering the corresponding consumable based on the order sheet for target laboratory.

6. The inventory alert system according to claim 5, further comprising,
an input device, wherein the control unit is configured to receive an input from the input device, the input being indicative of an accuracy of the calculation of an expected demand deviation and to adjust the exceptional deviation threshold EDT and/or the alert threshold AT and/or parameters of the function for calculation of the alert triggering factor F and/or parameters of the function for calculation of the expected demand deviation based on the input.

7. The inventory alert system according to claim 1, wherein the at least one laboratory instrument comprises at least one analytical instrument and wherein the at least one processing step of a biological sample comprises at least one diagnostic test of the biological sample for obtaining a measurement value from biological sample.

8. A computer implemented method for alerting laboratories, the method comprising:
communicatively connecting a plurality of laboratories to a control unit, wherein the plurality of laboratories comprise at least one triggering laboratory and at least one target laboratory, each laboratory comprising at least one laboratory instrument configured to carry out at least one processing step of a biological sample and a consumable required to carry out the processing step;
determining a usual demand level T of the consumable required to carry out a processing step by the corresponding triggering laboratory;
processing actual usage data X of the consumable required to carry out the processing step by the corresponding triggering laboratory;
determining a deviation D of the actual usage data X from the usual demand level T of the corresponding triggering laboratory;
calculating an alert triggering factor F of each deviation D that exceeds an exceptional deviation threshold EDT; and
alerting the target laboratory by the control unit when an aggregation Fₜₒₜ of each alert triggering factor F corresponding to each triggering laboratory in the proximity P of the target laboratory exceeds an alert threshold AT.

9. The method for alerting laboratories according to claim 8, wherein the usual demand level T of the consumable required to carry out the processing step is determined based on an average and/or median number of conducted and/or ordered processing step(s) per defined period corresponding to the respective triggering laboratory and/or determined based on historical data comprising one or more of: a seasonal variation, a schedule-based variation, an event-linked variation and a trend associated variation, and/or geographically influenced variation.

10. The method for alerting laboratories according to claim 8, wherein the alert triggering factor F is a function of one or more of: the usual demand level T, the deviation D, and the proximity P of the triggering laboratory and the target laboratory; and/or the proximity P is a weighted function of one or a combination of more of the following parameters: a geographical distance d1-dn between the triggering laboratory and the target laboratory, a travelling distance dt between the triggering laboratory and the target laboratory, a travelling time tt between the triggering laboratory and the target laboratory, a travelling frequency tf between the triggering laboratory and the target laboratory, and/or a selected geographical and/or political area A; the proximity function P having one or more of the following weights: a known infectious disease spread pattern overlapping the triggering laboratory and the target laboratory and/or a known major event with a known and/or expected elevated infectious disease risk level with known and/or suspected common participants from the geographical area of the triggering laboratory and the target laboratory.

11. The method for alerting laboratories according to claim 8, further comprising,
calculating an expected demand deviation of the consumable for the target laboratory, the expected demand deviation being a function of the usual demand level T of the target laboratory and/or a function of each alert triggering factor F corresponding to each triggering laboratory in the proximity P of the target laboratory and/or a function of the deviation D of each triggering lab.

12. The method for alerting laboratories according to claim 11, further comprising,
generating an order sheet of the corresponding consumable based on the expected demand deviation of the consumable for the target laboratory and the actual inventory of the consumable in the target laboratory and/or automatically ordering the corresponding consumable based on the order sheet for target laboratory.

13. The method for alerting laboratories according to claim 8, further comprising,
receiving an input from an input device, the input being indicative of an accuracy of the calculation of an expected demand deviation; and
adjusting the exceptional deviation threshold EDT and/or the alert threshold AT and/or parameters of the function for calculation of the alert triggering factor F and/or parameters of the function for calculation of the expected demand deviation based on the input.

14. The method for alerting laboratories according to claim 8, wherein the at least one laboratory instrument comprises at least one analytical instrument and wherein the at least one processing step of a biological sample comprises at least one diagnostic test of the biological sample for obtaining a measurement value from biological sample.

15. A computer program product having program code, in order to perform the method according to claim 8 when the program is executed on a computer or computer network.
